(19) United States
(12) Patent Application Publication
(10) Pub. No.: US 2016/0310525 A1
(43) Pub. Date: Oct. 27, 2016

(54) PREPARATION FOR PREVENTION AND/OR TREATMENT OF PERI-IMPLANTITIS

(71) Applicant: RLS GLOBAL AB, Göteborg (SE)

(72) Inventors: Ulrica ALMHÖJD, Bohus-Björkö (SE); Jan FORNELL, Stockholm (SE)

(21) Appl. No.: 15/102,168

(22) PCT Filed: Dec. 8, 2014
(86) PCT No.: PCT/EP2014/076909

§ 371 Date: Jun. 6, 2016

(30) Foreign Application Priority Data
Dec. 9, 2013 (SE) 1351468-2
Apr. 11, 2014 (SE) 1450452-6

(51) Int. Cl. A61K 33/20; A61K 9/06; A61K 9/00; A61K 47/02; A61K 47/22; A61K 31/198; A61K 47/38

(52) U.S. Cl. CPC A61K 33/20; A61K 31/198; A61K 9/06; A61K 47/38; A61K 47/02; A61K 47/22; A61K 9/0063

(57) ABSTRACT

The invention relates to a kit of parts for treatment and/or prevention of peri-implantitis. The kit of parts comprises a first aqueous component comprising one or more amino acids, and a second aqueous component comprising an active halogen compound. There is also provided a treatment preparation prepared from the components and uses thereof in the prevention and/or treatment of peri-implantitis.

### TECHNICAL FIELD

The present invention relates to a kit of parts and/or a preparation for use in prevention and/or treatment of peri-implantitis. Furthermore, the invention also relates to a method of using the kit and the preparation.

### BACKGROUND

Periodontal diseases and aplasia are common reasons why some people lack one or more teeth. For many years the only appliance for missing teeth was bridges. Bridges are composed of one or more artificial teeth which are attached firmly to neighboring natural teeth. This technique is excellent for replacing one or two teeth and has the advantage of being stable and not removable. Bridges are useful for replacing only the missing teeth, but cannot replace any missing bone or gum tissue. Their main disadvantage is that the teeth necessary to support the bridge may not be strong enough to bear any additional load, or they may not be in the right places. In addition, the aggressive grinding on the existing teeth which is necessary to attach the bridge is not desirable. Still a disadvantage of bridges is that thorough cleaning around the bridge is often difficult making it hard to keep good oral hygiene.

Dental implants are an attractive alternative to bridges and have been used for more than 20 years. The dental implant is a surgical device used to replace the one or more missing teeth. The dental implant comprises a part that is fused to the bone by so-called osseointegration. The dental implant may further support a crown or bridge. Thus, the dental implant may be seen as a way of providing an artificial tooth root to which a crown may be attached.

There are many advantages associated with dental implants. For instance, use of dental implants usually provides a good appearance and good comfort. Further, dental implants feel and function like a person's own teeth.

For a long time it was believed that dental implants were associated with no serious side effects. However, it is now realized that dental implants may cause diseases, infections and/or inflammations. Among these dental implant-related diseases, infections and/or inflammations so-called peri-implantitis and oral mucositis associated with a dental implant account for a large number of cases.

Peri-implantitis has been defined as a localized lesion involving bone loss around an osseointegrated implant. Peri-implantitis is a serious complication, and it is believed to occur in a large number of patients with dental implants. It has been reported that for patients having a dental implant one out of ten or even more suffer from peri-implantitis. Thus, it is clear that peri-implantitis is an increasing problem in modern dentistry.

Oral mucositis associated with a dental implant, which may also be denominated oral mucositis or peri-implant mucositis, is a reversible inflammatory change of the soft tissues surrounding the dental implant without concomitant bone loss. The prevalence of oral mucositis is difficult to estimate, but is believed to be higher than that of peri-implantitis. Generally, oral mucositis is a stage preceding peri-implantitis.

Features that contribute to making prevention and/or treatment of peri-implantitis difficult are the rough surface and/or the threads of the dental implants. The rough surface and the threads increase the dental implant surface area thereby improving osseointegration and healing after installation of the dental implant. However, in case of infection and/or microbial colonization a large dental implant surface area is a disadvantage since it allows for microorganisms to harbour in places that are difficult to see visually and/or reach with instruments and/or preparations thereby making microorganism eradication and killing difficult.

In Oral Health Journal, August 2013, Mark Nicolucci describes current treatments of peri-implantitis. It is disclosed that treatment of peri-implantitis may be approached like treatment of periodontitis. However, periodontitis and peri-implantitis do not respond to treatment similarly. Peri-implantitis lesions are said not to respond well to improved oral hygiene and professional cleanings as is highly effective with periodontitis.

Attempts to treat infected implant surfaces have been made. In the abstract of Arch Oral Biol. 2012 July; 57(7):940-7 it is disclosed that sodium hypochlorite was effective when tested as an antimicrobial agent on biofilm on titan specimens, but was not recommended for the topical disinfection and detoxification of infected implant surfaces due to possible toxicity and lack of broad-spectrum antimicrobial effect.

Prevention and/or treatment of peri-implantitis are commonly carried out in specialist clinics such as dentist specialist clinics. Frequently, the treatment is followed up in a generalist clinic. It would be desirable, however, if at least part of the prevention and/or treatment could take place in a generalist clinic or even in a patient's home since this is often more convenient for the patient. In particular, prevention of peri-implantitis would be desirable thereby avoiding or minimizing peri-implantitis.

Thus, there exists a need for methods allowing for prevention and/or treatment of peri-implantitis as well as for preparations for use in said methods.

It is an object of the present invention to fulfill said need and/or to overcome or at least mitigate some of the disadvantages associated with the prior art.

### DESCRIPTION

Accordingly, there is provided a kit of parts for use in the prevention and/or treatment of peri-implantitis. The kit of parts comprises
a) a first aqueous component comprising one or more amino acids, and
b) a second aqueous component comprising an active halogen compound. The pH of the first component and/or the second component may be from about 9 to about 11.5.
   The treatment of peri-implantitis may be surgical, i.e. surgical treatment of peri-implantitis.

Further, there is also provided the use of a kit of parts comprising
a) a first aqueous component comprising one or more amino acids, and
b) a second aqueous component comprising an active halogen compound for the manufacture of a medicament for the prevention and/or treatment of peri-implantitis. The pH of the first component and/or the second component may be from about 9 to about 11.5.

The treatment of peri-implantitis may be surgical, i.e. surgical treatment of peri-implantitis.

The kit of parts may further comprise instructions for use. The instructions may involve instructions for mixing the first aqueous component with the second aqueous component into a treatment preparation, how to apply the treatment preparation and/or how often the treatment preparation should be applied. The instructions may also include instructions for adding further components such as one or more compounds being a hydrazine derivative. The hydrazine derivative may be a compound of formula (I) as described herein.

As described herein, peri-implantitis is understood to be a localized lesion involving bone loss around an osseointegrated dental implant. Further, the peri-implant pockets and/or tissues surrounding the dental implant may be swollen and/or involved in inflammation and/or infection. Oral mucositis is understood to be a reversible inflammatory change of the soft tissues surrounding the dental implant without concomitant bone loss. Generally, peri-mucositis is a stage preceding peri-implantitis. In this document, the expressions “oral mucositis”, “oral mucositis associated with a dental implant” and “peri-implant mucositis” are used interchangeably.

The term “active halogen compound” herein refers to a halogen in the form of an ion, gas, salt or hypohalogenite of a halogen.

The two components of the kit of parts are intended to be mixed together and thereby form a treatment preparation for use in the prevention and/or treatment of peri-implantitis.

The first component reduces the aggressiveness of the active halogen compound to living tissues. The treatment preparation formed from the two components comprises one or more amino acids that provide less aggressive and toxic means for treating peri-implantitis and/or oral mucositis. The treatment with the treatment preparation utilizes the two liquid components which when combined to the treatment preparation and applied on a site of treatment such as a dental implant surface and/or lesions associated with peri-implantitis lead to a chemical reaction which is believed to result in primary halogenated amines in the form of (primary) halogenated amino acids such as primary chloroamino acids. By primary chloroamino acids is intended amino acids in which the amine carries one chlorine atom. The treatment disclosed herein is believed to utilize this chemical reaction.

The kit of parts for use in prevention of peri-implantitis described herein may be applied by administration of the first and second components once or several times. Prior to administration, the first and second components may be mixed to form a treatment preparation. Thus, the first and second components of the kit of parts may be administered to a treatment site repeatedly. For instance, examination of a treatment site such as tissues and/or peri-implant pockets will reveal if it is suitable to add the first and second components. The examination may be visual inspection. The examination may be made with respect to bleeding on probing, the degree of swelling of the tissues, colour of the tissues etc. The skilled person such as a dentist will be able to determine which time interval between treatments is appropriate, i.e. how often the first and second components should be applied. As an example, the time interval between treatments may be one, two, three, four, five, six weeks or more.

As used herein, the term “treatment” refers to treatment of peri-implantitis aiming at removal, eradication and/or killing of microorganisms such as bacteria in order to allow for osseointegration and healing. The treatment thus results in improved osseointegration and healing. As used herein, the term “osseointegration” is understood to mean the formation of a direct contact between the dental implant surface and bone. The term “prevention” refers to a measure taken in order to minimize and/or avoid peri-implantitis. Prevention may take place at a stage preceding peri-implantitis. Oral mucositisis is a stage preceding peri-implantitis, and treatment of oral mucositis is thus a way of preventing peri-implantitis.

It is particularly desirable to prevent peri-implantitis. Significantly, it has been found by the inventors of the present invention that prevention may be carried out using the treatment preparation described herein. As shown in the Examples, prevention using the treatment preparation Perisolv® or Perio+® was successful in preventing peri-implantitis. Similarly, oral mucositis could be treated using Perisolv® or Perio+®.

The uses of chloroamines as is known in the art commonly rely on the use of reactions that are subsequent to the first reaction, i.e. the reaction in which primary halogenated amines are formed, and in which the preparation should have a low pH, i.e. just below neutral pH such as pH about 6. These subsequent reactions result in dihalogenated amines, i.e. amines in which the nitrogen carries two chlorine atoms. The pH is then normally lower than a neutral pH, which is contrary to the present treatment and/or prevention with the treatment preparation disclosed herein wherein the pH is high and basic.

After being mixed the components will also have low impact on the environment, as the mixed components will decompose into salts, gases and water in nature. Attention is drawn to the fact the concentrations of halogen such as chlorine used in the components and treatment preparations are lower than the amounts of chlorine used in chlorination of water for purification thereof or in normal house hold bleach product containing chlorine.

The pH of the first and/or second component may be from 9 to 11, or from 9.5 to 11.5, or from 10 to 11.5, or from 10.5 to 11.5, or from 11 to 11.5, or from 9 to 10.5.

The treatment preparation may have antibacterial effects making it useful for the treatment and/or prevention of dental implant-related diseases, infections and inflammations such as peri-implantitis and oral mucositis.

The active halogen compound may be an active chlorine compound.

The active chlorine compound may be Cl₂, chloride, hypochlorite, chlorite, chlorate, perchlorate and/or a hypochlorite compound. Alternatively, the active chlorine compound may be Cl₂, hypochlorite, chlorite, chlorate, perchlorate and/or a hypochlorite compound. Further, the active chlorine compound may be Cl₂, hypochlorite or a hypochlorite compound. Further, the active chlorine compound may be hypochlorite or a hypochlorite compound.

The ion compounds may be in the form of sodium, calcium, lithium and/or potassium form, e.g. the hypochlorite compound may be sodium hypochlorite, calcium hypochlorite, lithium hypochlorite and/or potassium hypochlorite.

The amount of the active halogen compound in the second component may be 0.5-5, 0.5-3, or 1-2% (by weight).

If nothing else is mentioned, percentage by weight herein means to refer to the weight of a compound or the like relative the total amount of the mentioned component or the like comprising the compound. Percentage by weight is also denominated weight % or wt %. In this document, the expressions “% (by weight)”, “weight %” and “wt %” are used interchangeably. Alternatively, the concentration may be expressed as weight/weight % (w/w %). Since the first and second components are aqueous compositions the concentration may also be expressed as density using w/v, i.e. weight per volume.

Combining the second component including a halogen compound such as hypochlorite with the first component including one or more amino acids such as glutamic acid, leucine and lysine is effective for prevention and/or treatment peri-implantitis. It is also effective for treating oral mucositis.

Advantageously, it has been found that use of a treatment preparation as described herein will not adversely affect the surface of a titanium implant. This is a significant benefit, since the dental implant surface is generally designed to facilitate osseointegration and therefore should not be changed or damaged. Thus, use of the treatment preparation as described herein will have no or minimal negative impact on the osseointegration process of the implant.

A used herein, amino acids are organic compounds made from amine (—NH₂) and carboxylic acid (—COOH) functional groups, along with a side-chain specific to each amino acid.

The one or more amino acids may be selected from the group consisting of alanine, arginine, asparagine, aspartic acid, glutamic acid, glutamine, isoleucine, leucine, lysine and/or valine.

These amino acids have been identified to be of the kind to form primary halogenated amino acids such as primary chloroamino acids. The pK values and/or properties of the side chains are such that the primary halogenated amino acids may be formed and uses thereof also cause low risk, if any, of adverse effects on a subject such as a patient.

The one or more amino acids may comprise or consist of glutamic acid, leucine and lysine.

According to an embodiment, the first component may comprise 0.1-1% (by weight) of said one or amino acids.

The amino acid concentration in the first component may be 0.1-1%, 0.4-1.0% or 0.5-1% (by weight), or 0.4-0.8% or 0.5-0.8% (by weight). The amino acid composition may contain a mixture of amino acids mixed in a relation by weight between each amino acid of about 2:1 to 1:2, preferably about 1:1.

The two components may be mixed in the volume ratios of 1:2 to 2:1, preferably about 1:1, thereby forming the treatment preparation for use in the treatment and/or prevention of an implant-related disease, infection and/or inflammation such as peri-implantitis and/or oral mucositis. At room temperature (20 degrees Celsius) and 1 atm of pressure (101325 Pa), and within 30 seconds after the components have been mixed together, the treatment preparation has a pH of from 9 to 11.5, or from 9 to 11, or from 9.5 to 11.5, or from 10 to 11.5, or from 10.5 to 11.5, or from 11 to 11.5, or from 9 to 10.5, or from 9.5 to 10.5.

The treatment preparation described herein may be applied once or several times during the same treatment session and/or during separate treatment sessions. Thus, the treatment preparation may be administrated to a treatment site repeatedly. Before a new treatment session takes place, examination of a treatment site such as tissues and/or peri-implant pockets will reveal if it is suitable to add the treatment preparation. The examination may be visual inspection. The examination may be made with respect to bleeding on probing, the degree of swelling of the tissues, colour of the tissues etc. The skilled person such as a dentist or a dental hygienist will be able to determine which time interval between treatment sessions is appropriate, i.e. how often the treatment preparation should be applied. As an example, the time interval between treatment sessions may be one, two, three, four, five, six weeks or more. Generally, treatment of peri-implantitis with the treatment preparation described herein takes place only once whereas prevention of peri-implantitis may comprise application of the treatment preparation during one or several treatment sessions.

The treatment preparation described herein facilitates removal of undesired matter thereby shortening the treatment time. Preferably, the steps are repeated during the treatment session so that the treatment preparation is applied several times.

In this way there is provided a way of securing that the reaction forming the primary halogenated amino acids have the proper reaction condition to occur on the treatment site. The treatment site may be a dental implant, a peri-implant pocket, a peri-implantitis lesion and/or tissues associated with peri-implantitis or oral mucositis. Thus, the provision of a kit of parts provides means to control that the desired reaction occurs on the treatment site.

The use may involve applying the treatment preparation before or during the appearance of gas bubbles from the treatment preparation. If an active chlorine compound is used, a smell of chlorine also appears along with the gas bubble formation. In this way, the occurrence of the proper reaction is secured.

The first aqueous component may further comprise a gel substance.

The gel substance provides moist keeping means to keep a moist environment for the treatment site, and in particular the gel substance reduces evaporation from the aqueous treatment preparation prepared from the first and second components, when applied to the treatment site. Furthermore, the gel substance also provides a proper consistency to the treatment preparation prepared from the two components.

A treatment preparation provided with a gel substance and having the “high” basic pH provides the use for effective treatment and/or prevention as mentioned herein above, while providing low aggressiveness to a treatment site.

The gel substance may comprise or consist of polyethylene glycol (PEG), and/or carboxymethyl cellulose and/or a polysaccharide substance or a salt thereof, such as sodium carboxymethyl cellulose (Na-CMC).

Such a substance provides said consistency and acts as a moist keeping substance preventing said evaporation from the treatment preparation and treatment site.

The first component may comprise 2-4% (by weight) gel substance.

The first component may further comprise TiO₂ and/or NaCl.

The addition of NaCl adds to the active chlorine compound content present in the first component and treatment preparation. NaCl also has an antibacterial effect as such.

The components may be the ones sold under the trademark Carisolv®, Perisolv® or Perio+® (RLS Global AB).

Carisolv®, Perisolv® or Perio+® may have the compositions indicated in Table I. In other words, Carisolv®, Perisolv® or Perio+® may have the following compositions.

The preparation Carisolv® is an aqueous composition comprising a first aqueous component comprising a mixture of glutamic acid, leucine and lysine (0.1-1 wt %), NaCl (0.3-0.6 wt %) and sodium carboxymethyl cellulose gel (2-4 wt %), and a second aqueous component comprising NaOCl (1-2 wt %). The first component and the second component may be added in an amount providing a pH from 9 to 11.5.

The preparation Periosolv® or Perio+® is an aqueous composition comprising a first aqueous component comprising a mixture of glutamic acid, leucine and lysine (0.1-1 wt %), TiO₂ (0.03-0.1 wt %), NaCl (0.3-0.6% wt %) and sodium carboxymethyl cellulose gel (2-4 wt %), and a second aqueous component comprising NaOCl (1-2 wt %). The first component and the second component may be added in an amount providing a pH from 9 to 11.5.

The clinical effectiveness with respect to prevention and/or treatment of peri-implantitis may be further enhanced by subjecting the treatment preparation as described herein, wherein said treatment preparation includes TiO₂, to irradiation from 10 to 700 nm, or from 200 to 400 nm or from 400 to 700 nm. In this document, the term “nm” stands for nanometer. While not wishing to be bound by any specific theory, it is believed that this enhanced clinical effect may be due to the formation of singlet oxygen. Previously, it has been reported that singlet oxygen produced by irradiation of TiO₂ may be involved in oral bacterial disinfection (J. Clin. Biochem. Nutr., September 2012, Vol. 51, No. 2, pages 128-131.)

The kit of parts may therefore further comprise an instrument able to operate at wavelengths such as ultraviolet (uv) wavelengths. The instrument may be operated at from 10 to 700 nm, or from 200 to 400 nm or from 400 to 700 nm, and may be used to irradiate the treatment preparation described herein

A major difficulty when cleaning and/or treating dental implants is to remove all or substantially all undesired matter such as infected tissue on the implant surface. Detection of infected tissue may be facilitated by staining of said infected tissue using a hydrazine derivative. Advantageously, staining allows visual detection of undesired matter such as infected tissue on a dental implant surface.

The kit of parts may therefore further comprise one or more hydrazine compounds of formula (I):

RNHNH₂ (I)

The R group of the hydrazine compound RNHNH₂ is a chromophore or forms a chromophore with NHNH₂.

In this document, the term “chromphore” means the part of the molecule resulting in its colour. The colour arises when that part of the molecule absorbs certain wavelengths of light such as visible light and transmits or reflects others. In this document, visible light is defined as electromagnetic radiation having a wavelength in the range of about 380 nm to about 750 nm. Detection may be made without or with the aid of an optical instrument.

It is to be understood that the hydrazine derivative of formula (I) may be, for instance, a carbazide or a hydrazide.

Examples of hydrazine derivatives of formula (I) that may be used include compounds of formula (Ia), (Ib), (Ic) and (Id) below.

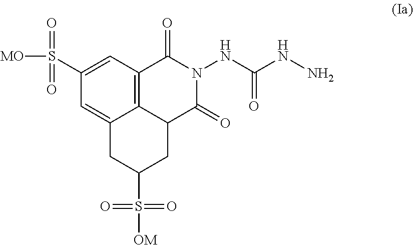

wherein M represents a monovalent metal ion selected from Li⁺, K⁺ and Na⁺. When M is K⁺ the compound of formula (Ia) is denominated Lucifer Yellow.

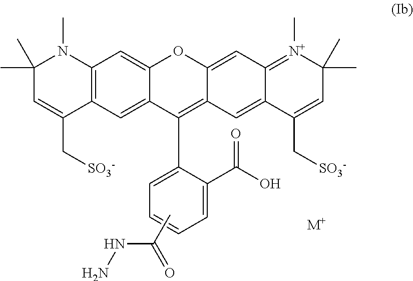

wherein M⁺ represents a monovalent metal ion selected from Li⁺, K⁺ and Na⁺.

The trade name of the compound of formula (Ib) when M⁺ is Na⁺ is Alexa Fluor® 594 hydrazide sodium salt.

The chemical name for the compound of formula (Ib) is 6-(2-carboxy-5-(hydrazinecarbonyl)phenyl)-1,2,2,10,10,11-hexamethyl-2,10-dihydro-1H-pyrano[3,2-g]diquinoline-11-ium-4,8-diyl)methanesulfonate.

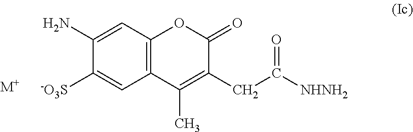

wherein M⁺ represents a monovalent metal ion selected from Li⁺, K⁺ and Na⁺.

The trade name of the compound of formula (Ic) when M⁺ is Na⁺ is Alexa 350.

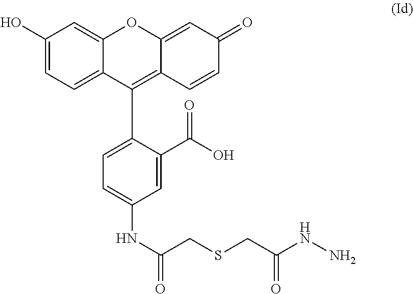 [SMILES: NNC(=O)CSCC(=O)Nc1ccc(-c2c3ccc(=O)cc-3oc3cc(O)ccc23)c(C(=O)O)c1]

The trade name of the compound of formula (Id) is 5-(((2-(carbohydrazino)methyl)thio)acetyl)aminofluorescein, whereas the chemical name suggested by Chemdraw (CS Chemdraw Ultra, Cambridge Soft, USA) for the compound (Id) is 5-(2-2-hydrazinyl-2-oxoethylthio)acetamido)-2-(3-hydroxy-6-oxo-6H-xanthen-9-yl) benzoic acid.

It will be appreciated that the hydrazine derivative mentioned herein may be mixed with one or more other hydrazine derivatives. Further, the hydrazine derivative may be mixed with other dyes such as Patent Blue V. Patent Blue may exist as its calcium salt and has CAS number 3536-49-0.

Further examples of hydrazine derivatives include HiLyte Fluor™ 488 hydrazide, HiLyte Fluor™ 555 hydrazide, HiLyte Fluor™ 594 hydrazide, HiLyte Fluor™ 647 hydrazide, HiLyte Fluor™ 680 hydrazide

In order to ensure that all or essentially all undesired matter such as infected tissue is removed, eradicated and/or killed a solution of a hydrazine derivative may be added to the implant thereby staining any present infected tissue. If infected tissue is present, they may easily be detected by staining and subsequently removed. The infected tissue is believed to contain bacteria. While not wishing to be bound by any specific theory, it is believed that bacteria contain ester groups and that staining may take place upon reaction of these ester groups with the hydrazine derivative.

The hydrazine derivative may be added to the implant surface followed by addition of the treatment preparation as described herein. For instance, an aqueous solution of the hydrazine derivative may be provided to the implant surface followed by addition of the treatment preparation described herein. Rinsing with an aqueous solution may take place after addition of the hydrazine derivative to the implant surface. Alternatively, the hydrazine derivative may be mixed with the treatment preparation. In the latter case, staining may be visualized after rinsing and then further treatment preparation may be added to the stained parts of the implant surface. Removal of the infected tissue may be further improved by mechanical treatment of the implant surface. For this purpose, a dental scrape instrument may be used.

There is also provided a treatment preparation for use in prevention and/or treatment of peri-implantitis, wherein the treatment preparation is obtainable by mixing a first and a 10 second component as described herein. The first aqueous component and the second aqueous component may be mixed in a volume ratio of 1:2 to 2:1 or in a volume ratio of about 1:1. The prevention may include treatment of peri-mucositis.

Such a treatment preparation has shown the desired effects in uses described herein.

There is also provided the use of a treatment preparation for the manufacture of a medicament for prevention and/or treatment peri-implantitis,

wherein the treatment preparation is obtainable by mixing a first and a second component as described herein. The first aqueous component and the second aqueous component may be mixed in a volume ratio of 1:2 to 2:1 or in a volume ratio of about 1:1.

The treatment preparation may have a pH of about 9 to 11.5 within 30 seconds after the components have been mixed together (at room temperature and 1 atm of pressure (101325 Pa)). The pH may be from 9 to 11 or from 9 to 10.5, or from 9.5 to 10.5, or from 9.5 to 11.5, or from 10 to 11.5, or from 10.5 to 11.5, or from 11 to 11.5.

This provides the means for the proper reaction to occur on the treatment site such as an implant surface, peri-implant pockets and/or tissue associated with the dental implant described herein, while providing all the above-mentioned advantages.

As mentioned herein, the use of a treatment preparation may involve applying the treatment preparation to a treatment site. The treatment preparation may be applied before or during formation of gas bubbles appearing from the treatment preparation. If an active chlorine compound is used, a smell of chlorine also appears along with the gas bubble formation. In this way, the occurrence of the proper reaction is secured.

There is also provided a method for treatment of peri-implantitis, said method comprising the step of applying a treatment preparation as described herein to a treatment site. The treatment site may be a dental implant surface, peri-implant pockets and/or tissue surrounding a dental implant.

The treatment preparation and/or decomposed parts from the treatment site may then be removed from the treatment site.

Prior to applying the treatment preparation, the tissue area of the treatment site may be opened and/or made accessible by, for example, a flap elevation or raising of buccal and palatinal muco-periost flaps as is known in the art. The method for treatment of peri-implantitis described herein may thus be a surgical method. The treatment site may be a lesion and/or a dental implant associated with peri-implantitis.

The method for treating peri-implantitis may comprise the steps of:
opening of the tissue area of the treatment site by surgical means,
applying a treatment preparation to the treatment site, the treatment preparation comprising a first component with one or more amino acids and a second component with an active halogen compound;
removing the treatment preparation and/or decomposed parts from the treatment site.

The removal may include removal using an ultrasonic device.

The steps of the method may be repeated once or several times as considered appropriate by a skilled person such as a dentist. The treatment preparation may be allowed to incubate so as to allow the treatment preparation to act on the treatment site and to decompose parts present therein. Accordingly, the method for treating peri-implantitis may comprise the steps of:
opening of the tissue area of the treatment site,
applying a treatment preparation to the treatment site, the treatment preparation comprising a first component with one or more amino acids and a second component with an active halogen compound;
optionally incubating the treatment preparation applied in the step above so as to allow the treatment preparation to act on the treatment site and to decompose parts present therein;
removing the treatment preparation and/or decomposed parts from the subject; and
optionally, repeating the steps above.

The removal may include removal using an ultrasonic device. The treatment preparation facilitates removal thereby shortening the treatment time. Preferably, the steps are repeated during the treatment session so that the treatment preparation is applied several times.

In order to enhance the clinical effectiveness of the treatment preparation, wherein the treatment preparation includes TiO₂, the treatment preparation may be subjected to irradiation from 10 to 700 nm, or from 200 to 400 nm or from 400 to 700 nm. The treatment preparation may be subjected to irradiation before and/or after application to a treatment site. While not wishing to be bound by any specific theory, it is believed that this enhanced clinical effect may be due to the formation of singlet oxygen. Thus, the method for treating peri-implantitis may further comprise the step of:
subjecting the treatment preparation to irradiation from 10 to 700 nm, or from 200 to 400 nm or from 400 to 700 nm.

To further enhance the treatment, mechanical instrumentation may be used to remove, eradicate and/or kill microorganisms such as bacteria involved in peri-implantitis. Particularly, mechanical instrumentation may aid in removing bacteria on the dental implant surface resulting in a cleaned implant surface. Thus, the method for treating perk implantitis may further comprise a step of:
subjecting at least part of the treatment site to mechanical treatment.

In order to improve detection of infected tissue on the dental implant and/or tissue surrounding the implant, the method above may include a step involving staining the infected tissue using a hydrazine derivative as described herein. Thus, the method may further comprise a step of:
applying a hydrazine derivative to the treatment site. The hydrazine derivative may be a compound of formula (I) as described herein. Further, the hydrazine derivative may be provided as an aqueous solution, and rinsing with an aqueous solution such as water may take place after application of the hydrazine derivative.

An additional surgical intervention may also be provided to improve access and treatment of the tissue associated with the peri-implantitis, thereby also providing a stabilization and regeneration of the treatment site during healing, including regeneration of bone formation. The surgical intervention may also provide an additional step of removing the dead tissue, inflammatory tissue or the like. It is an advantage of the treatment preparation that is capable of decomposing dead tissue, inflammatory tissue and/or the like, and that it aids in the removal of such tissue as well as pus, bacteria and the like.

Accordingly, the decomposing step by the use of the treatment preparation provides a debridement at the treatment site and thus removal of non-desired tissue or other non-desired parts from the treatment site.

The treatment preparation may therefore be used to provide the debridement action, in particular when the preparation has a pH of 9 to 11.5 when applied to the treatment site.

Of course, the best option is to prevent peri-implantitis from occurring. It has been found that the treatment preparation described herein may be successfully used for such prevention. In particular, it has been found that the preparation Perisolv® or Perio+® is successful in preventing peri-implantitis. Similarly, it has been found that the preparation Perisolv® or Perio+® is successful in treating oral mucositis. Since oral mucositis is a stage preceding peri-implantis, and which may develop into peri-implantitis if left untreated, it is considered that treatment of oral mucositis is a way of preventing peri-implantitis.

Accordingly there is provided a method for preventing peri-implantitis, wherein the treatment preparation is obtainable by mixing a first and a second component as described herein. The method comprises the step of:
applying the treatment preparation to a treatment site such as tissues surrounding the dental implant and/or peri-implant pockets.

After application, the treatment preparation may be allowed to incubate. The treatment preparation may be removed by rinsing with an aqueous solution such as water or saline. The treatment preparation may be applied once or several times. The skilled person such as a dentist will be able to judge how often the treatment preparation should be applied. For instance, examination of tissues and/or per-implant pockets will reveal if it is suitable to add the treatment preparation. The treatment preparation may be applied repeatedly with a time interval of one, two, three, four, five, six weeks or more.

The method for preventing peri-implantitis may comprise treatment of oral mucositis.

In order to further enhance the clinical effectiveness of the treatment preparation including TiO₂, the treatment preparation may be subjected to irradiation from 10 to 700 nm, or from 200 to 400 nm or from 400 to 700 nm. The treatment preparation may be subjected to irradiation before or after being applied to the treatment site. While not wishing to be bound by any specific theory, it is believed that this enhanced clinical effect may be due to the formation of singlet oxygen. Thus, the method for preventing peri-implantitis may further comprise the step of:
subjecting the treatment preparation to irradiation from 10 to 700 nm, or from 200 to 400 nm or from 400 to 700 nm.

Mechanical instrumentation may be used to further improve removal, eradication and/or killing of any undesired matter such as bacteria. Thus, the method for preventing perk implantitis may further comprise the step of:
mechanically treating the implant and/or tissue surrounding the implant.

Rinsing with an aqueous solution such as water or saline may take place before, during and/or after the mechanical treatment.

In order to ensure that all or essentially all undesired matter such as infected tissue is removed from the implant surface, detection of said infected tissue may take place by staining with a hydrazine derivative. The hydrazine derivative may be as described in this document. Thus, the method for preventing peri-implantitis may further comprise the step of:
subjecting the implant and/or tissue surrounding the implant to a hydrazine derivative.

The hydrazine derivative may be provided as an aqueous solution, and a further step including rinsing with an aqueous solution such as water or saline may be performed. The staining may be established by visual inspection with or without an optical instrument.

The hydrazine derivative may also be used to detect a possible presence of infected tissue on an implant such as an implant surface. Thus, there is provided a method for detecting a possible presence of undesired matter such as infected tissue on a dental implant surface, said method comprising the step of:
adding a hydrazine derivative to a dental implant surface.

The hydrazine derivative may be provided as an aqueous solution, and a further step including rinsing with an aqueous solution such as water or saline may be performed. The method may further comprise a step of visual inspection to determine if staining has taken place. The visual inspection may take place without or with the aid of an optical instrument.

The methods concerning treatment and/or prevention of peri-implantitis described herein provide cleaning and/or treatment of the dental implant, the peri-implant pockets and/or tissue surrounding the dental implant thereby allowing for a better regeneration of the area treated, such as bone regeneration.

As mentioned herein the methods also provide means for antibacterial effects making it useful for the prevention and/or treatment of implant-related diseases, infections and/or inflammations.

The methods described herein utilize two liquid components which when combined and applied on an area of treatment leads to a chemical reaction resulting in, for example, primary halogenated amino acids. The components and mixtures are described herein.

The treatment preparation may be mixed prior to the application, wherein the resulting mixture, i.e. the treatment preparation, may applied to the treatment site, e.g. within seconds up to one minute. Incubation with the treatment preparation may then be allowed to proceed so as to, for example, separate dead tissue from healthy tissue as well as decomposing and/or destroying bacteria. The mixture in the form a solution may be left within the treatment area for, for example, 5 minutes or less, wherein bubbles and a smell of chlorine appear and then disappear in the preparation.

The treatment preparation used in the method for preventing peri-implantitis described herein may be applied once or several times during the same treatment session and/or during separate treatment sessions. Thus, the treatment preparation may be administered to a treatment site repeatedly. Before a new treatment session takes place, examination of a treatment site such as tissues and/or peri-implant pockets will reveal if it is suitable to add the treatment preparation. The examination may be visual inspection. The examination may be made with respect to bleeding on probing, the degree of swelling of the tissues, colour of the tissues etc. The skilled person such as a dentist or a dental hygienist will be able to determine which time interval between treatment sessions is appropriate, i.e. how often the treatment preparation should be applied. As an example, the time interval between treatment sessions may be one, two, three, four, five, six weeks or more.

The methods described herein utilize two liquid components which when combined and applied on an area of treatment leads to a chemical reaction resulting in, for example, primary halogenated amino acids. The components and mixtures are described herein.

In a step following the application of the treatment preparation, and possible incubation and removal thereof, the treatment site may be closed by flap closuring, e.g. by any method as in known in the art for flap closuring. For example, a bone cement, a biomembrane and/or a collagen layer may be applied around the treated lesion and implant from which the treatment preparation have been removed, and followed by a flap closure by a suture, e.g. by using monofilament suture closure procedure.

The methods may also include that the treatment preparation may be applied with a syringe or the like. The step of removal of the treatment preparation may also be carried out by use of a syringe.

### BRIEF DESCRIPTION OF THE DRAWINGS

FIG. 1 shows an X-ray photograph of a dental implant involved in peri-implantitis. The threads have been filled in to enhance visibility.

FIG. 2 is an X-ray photograph of the healed dental implant after one year. In FIG. 2 the threads have been filled in to enhance visibility.

The invention is further illustrated by the following non-limitative Examples.

### EXAMPLES

### Example 1

In Table I an example of the content of the two components as part of a kit for providing the treatment preparation is provided. The contents correspond in large to the components and preparations sold under the trademarks Carisolv® and Perisolv®.

TABLE I
Contents of component 1 and component 2 to be mixed with each
other just before application on a treatment site of a subject.
(water is present up to 100% by weight)

| | Component 1 | Component 2 |
|---|---|---|
| | (% by weight) | (% by weight) |
| NaOCl | — | ≦1-2 |
| Amino acids Lys, | 0.4-0.8* | — |
| Glu and Leu | | |
| TiO₂ | 0/0.03-0.1 | — |
| NaCl | 0.3-0.6 | — |
| Na-CMC (high viscosity/ | 2-4 | — |
| medium viscosity) | | |
| NaOH | Added in an amount | Added in an amount |
| | providing a pH | providing a pH from 9 to |
| | from 9 to 11.5 | 11.5 |

*total amount of amino acids, the amino acids being present in a relation by weight of about 1:1:1.

In Example 2 and Example 3 below the numbering of the teeth follows the Universal Numbering System. The treatments were performed by an experienced dentist consultant in periodontology. The preparation Perisolv® was freshly prepared prior to use.

### Example 2

### Surgical Treatment of Peri-Implantitis

The patient was a male born in 1943 who was on medication for high blood pressure and high levels of cholesterol. The patient was a nonsmoker. He had a dental implant in position 25. The dental implant was from the company Straumann, with seven threads (4.1×10 mm). In this document, the term “mm” stands for millimeter. The dental implant had been installed in 2007.

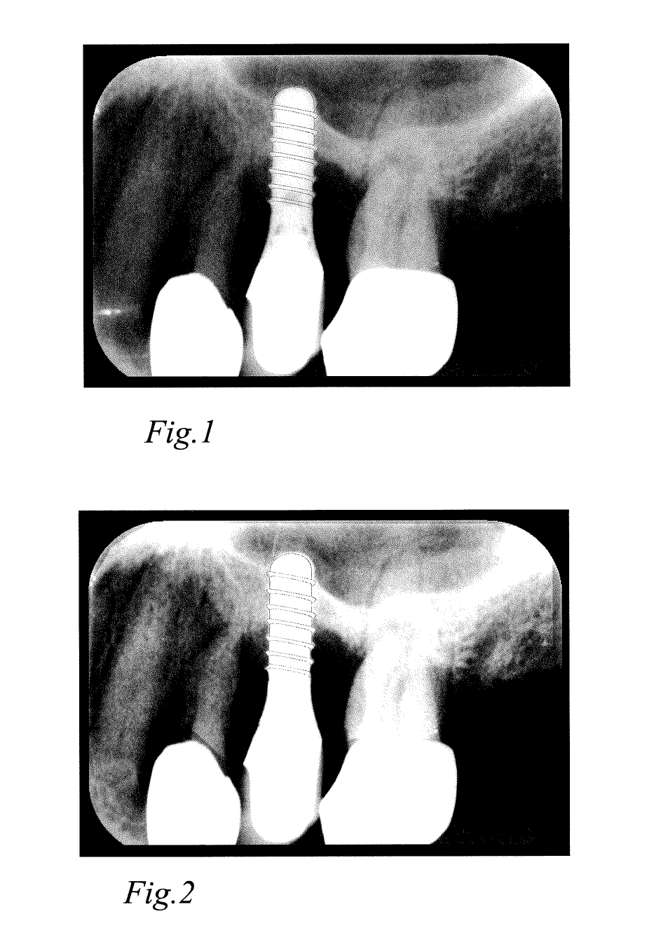

The patient was diagnosed with peri-implantitis in 2009. Clinical investigation revealed that an infection was present. X-ray analysis showed that the dental implant was shaded, which is interpreted as an infection. FIG. 1 shows an X-ray photograph of the dental implant involved in the peri-implantitis. In FIG. 1 the threads have been filled in to enhance visibility. The patient was treated as follows.

The patient was operated. Anesthetics with xylocaine-adrenaline was given to the patient. Buccal and palatinal muco-periost flaps were raised. Perisolv® was applied onto the surface of the dental implant and left for 30 seconds. Mechanical cleaning was then performed using an ultrasonic device with curettage of granulation tissue. Rinsing with water took place continuously during the mechanical cleaning. Perisolv® was then applied again onto the dental implant surface and surrounding tissues. Then, ultrasonic treatment in combination with rinsing with water took place. Due to the bone loss associated with the peri-implantitis the top four threads of the dental implant were not connected to bone. Bone ceramics from the company Straumann was therefore added around the dental implant in such a way that it was filled into the bone defect. Flap closure was performed using a suture closure procedure.

The patient was prescribed the antibiotic Azytromax, 500 mg, 6 tablets of which 2 should be taken the first day and thereafter one tablet per day. The patient was also prescribed mouth rinsing with chlorhexidine twice a day for a week.

The patient came back to the clinic for check-ups and X-ray analysis one week, three months, one year, two years and four years later. Complete healing of the peri-implantitis was observed one year after the operation had taken place, and the healing still remained after four years. All treads of the dental implant were covered by bone. X-ray analysis showed that the dental implant was no longer shaded, which means that complete healing has taken place. No inflammation was noticed. FIG. 2 is an X-ray photograph of the healed dental implant after one year. In FIG. 2 the threads have been filled in to enhance visibility.

### Example 3

### Prevention of Peri-Implantitis and Treatment of Oral Mucositis

The patient was a female born in 1949. The patient was a non-smoker. She was healthy, lacked tooth 11, and in 1999 she had a dental implant installed in position 11. The patient payed regular visit to a dental hygienist and during one of the routine check-ups inflammation around the dental implant was found, i.e. oral mucositis was diagnosed. The oral mucosa around the implant was swollen, pus was present and there was bleeding on probing. The peri-implant pocket depth around the dental implant was considered to be pathologic. X-ray analysis showed that the bone around the dental implant was slightly lower than normal. The bone lowering did not reach the first implant thread, though. The dentist examined the patient and interpreted the situation as a stage preceding peri-implantitis, said stage involving mucositis and slight bone resorption. The dentist then treated the patient as described below.

Perisolv® was applied for 30 seconds in the peri-implant pocket surrounding the dental implant. Mechanical treatment using an ultrasonic device and curettage of granulation tissue took place. The procedure was repeated once more. At the end, rinsing with water took place. The treatment took approximately 15 minutes. Three weeks later the patient came for a check-up. The tissue surrounding the dental implant looked healthy and normal. X-ray analysis showed that the bone close to the dental implant had started to heal.

It was concluded that the treatment with Perisolv® was successful in preventing peri-implantitis and in treating oral mucositis.

### Example 4

This experiment was performed in order to investigate if remnants of tissue on an implant neck could be detected by staining with a hydrazine derivative. The hydrazine derivative Lucifer Yellow was chosen, since it will exhibit fluorescence when irradiated with a suitable lamp.

An extracted dental implant from a patient suffering from peri-implantitis was received. The patient had received no treatment for peri-implantitis and the implant neck was thus provided with tissue from the patient. The implant was subjected to an aqueous solution of the hydrazine derivative Lucifer Yellow in a concentration of 15 mM. In this document, mM stands for millimolar, i.e. millimoles per liter. After rinsing with water the implant neck was subjected to irradiation from an ordinary dental lamp operating between 400 and 500 nm in order to see if fluorescence could be detected. Fluorescence was observed. A photograph was taken under a microscope, and it was concluded that tissue remained on the implant neck. The tissue was then cleaned with Carisolv® together with a brush. The cleaning was followed by rinsing with water. Visual inspection with the aid of the lamp revealed staining of some parts. Thus, some tissue remained on the implant neck. Cleaning was repeated with Carisolv® until no fluorescence was detected.

It was concluded that hydrazine derivatives allowed for detection of tissue on a dental implant.

1.-54. (canceled)

55. A method for treatment and/or prevention of peri-implantitis in a patient, wherein said method comprises applying a treatment preparation to a treatment site of the patient, said treatment preparation being obtained by mixing:
a) a first aqueous component comprising one or more amino acids selected from the group consisting of alanine, arginine, asparagine, aspartic acid, glutamic acid, glutamine, isoleucine, leucine, lysine and/or valine, and
b) a second aqueous component comprising an active chlorine compound selected from the group consisting of Cl₂, hypochlorite, chlorite, chlorate, perchlorate and/or a hypochlorite compound,
wherein the first aqueous component and/or the second aqueous component has a pH from 9 to 11.5,
thereby treating and/or preventing peri-implantitis in the patient.

56. The method according to claim 55, rein said method treats peri-implantitis in the patient.

57. The method according to claim 56, wherein the treatment of peri-implantitis comprises surgical treatment of peri-implantitis.

58. The method according to claim 55, wherein said method prevents peri-implantitis in the patient.

59. The method according to claim 58, wherein said method comprises treatment of oral mucositis.

60. The method according to claim 55, wherein the amount of the active chlorine compound in the second aqueous component is 0.5-5 wt %.

61. The method according to claim 55, wherein said one or more amino acids comprise glutamic acid, leucine, and lysine.

62. The method according to claim 55, wherein the first aqueous component comprises 0.1-1 wt % of said one or more amino acids.

63. The method according to claim 55, wherein the first aqueous component further comprises a gel substance.

64. The method according to claim 63, wherein the gel substance comprises sodium carboxymethyl cellulose (Na-CMC).

65. The method according to claim 64, wherein the first aqueous component comprises 2-4 wt % of the gel substance.

66. The method according to claim 55, wherein said treatment preparation is obtained by mixing:
a. a first aqueous component comprising 0.1 to 1 wt % of glutamic acid, leucine and lysine and 2-4 wt % of sodium carboxymethyl cellulose, and
b. a second aqueous component comprising 1-2 wt % of sodium hypochlorite.

67. The method according to claim 66, wherein said treatment preparation further comprises TiO₂ and/or NaCl.

68. The method according to claim 55, wherein said method further comprises contacting the treatment site with one or more compounds of a hydrazine derivative of formula (I)
RNHNH₂ (I)
wherein R is a chemical group containing a chromophore or forming a chromophore with NHNH₂, thereby staining infected tissue at the treatment site.

69. The method according to claim 55, wherein said treatment preparation is obtained by mixing:
a. a first aqueous component comprising:
0.4 to 0.8 wt % of glutamic acid, leucine and lysine, said amino acids being present in a relation by weight of 1:1:1,
2-4 wt % of sodium carboxymethyl cellulose,
from 0.03 to 0.1 wt % of TiO₂,
0.3 to 0.6 wt % of NaCl, and
NaOH added in an amount providing a pH from 9 to 11.5,
b. a second aqueous component comprising 1-2 wt % of sodium hypochlorite, and NaOH added in an amount providing a pH from 9 to 11.5.

70. The method according to claim 55, wherein the treatment site is a dental implant surface, a peri-implant pocket, or tissue surrounding a dental implant, or a combination thereof.

71. The method according to claim 55, wherein the first aqueous component and the second aqueous component are provided in a kit of parts, wherein the kit of parts further optionally comprises a hydrazine derivative of formula (I)
RNHNH₂ (I)
wherein R is a chemical group containing a chromophore or forming a chromophore with NHNH₂.

72. The method according to claim 71, wherein the kit of parts further comprises instructions for how to apply the treatment preparation, how often the treatment preparation should be applied, or for adding a hydrazine derivative of formula (I), or a combination thereof, wherein formula (I) is
RNHNH₂ (I)
wherein R is a chemical group containing a chromophore or forming a chromophore with NHNH₂.

73. The method according to claim 55, wherein the treatment preparation obtained by mixing said first aqueous component and said second aqueous component at room temperature and at 1 atm of pressure within 30 seconds after mixing has a pH from 9 to 11.5.

74. The method according to claim 55, wherein the first aqueous component and the second aqueous component are mixed in a volume ratio from 1:2 to 2:1.
